(19) United States
(12) Patent Application Publication
(10) Pub. No.: US 2013/0231560 A1
(43) Pub. Date: Sep. 5, 2013

(54) BLOOD FLOW MEASURING APPARATUS AND BLOOD FLOW MEASURING METHOD

(71) Applicant: NIHON KOHDEN CORPORATION, Tokyo (JP)

(72) Inventors: Shinya Nagata, Tokyo (JP); Takao Miyazaki, Tokyo (JP)

(73) Assignee: NIHON KOHDEN CORPORATION, Tokyo (JP)

(21) Appl. No.: 13/778,830

(22) Filed: Feb. 27, 2013

(30) Foreign Application Priority Data
Mar. 2, 2012 (JP) 2012-046368

(51) Int. Cl. A61B 5/0265

(52) U.S. Cl. CPC A61B 5/0265
USPC 600/430; 600/407

(57) ABSTRACT

A blood flow measuring apparatus includes: a module which is configured to emit radiation to an object to be measured and receive reflection of the radiation, to detect movement of the object to be measured based on a Doppler effect; a reflective holding member which internally hold the module with a gap from an abutment surface that is to be butted against a skin, and which includes a reflective member covering the module and configured to reflect the radiation; and a process circuit which is configured to receive an output from the module, and which is configured to perform at least a process related to a blood flow speed.

### CROSS-REFERENCE TO RELATED APPLICATION(S)

This application is based upon and claims the benefit of priority from prior Japanese patent application No. 2012-046368, filed on Mar. 2, 2012, the entire contents of which are incorporated herein by reference.

### BACKGROUND

The presently disclosed subject matter relates to an apparatus and method for measuring a blood flow.

In a measurement of a blood flow, an electromagnetic blood flow meter, an ultrasonic Doppler blood flow meter, or the like is used. In an electromagnetic blood flow meter, an exciting coil and electrodes are disposed in the periphery of a blood vessel, and an electro motive force caused by a blood flow which crosses the magnetic field produced by the exciting coil is measured to determine the blood flow volume.

In an ultrasonic Doppler blood flow meter, an ultrasonic wave is radiated to a blood flow, and the flow speed is measured based on the frequency change of a reflected wave of the radiated wave.

Also, JP-A-2003-79589 discloses an apparatus which radiates light or a microwave to measure the blood flow.

In an electromagnetic blood flow meter, there is a problem in that a probe of the blood flow meter must be attached to a blood vessel, and a large burden is placed on the subject. In an ultrasonic Doppler blood flow meter, a sensor must be press-attached to the skin. Also in this case, the burden on the subject is large.

The apparatus disclosed in JP-A-2003-79589 has a casing for holding a probe with being separated from the skin by a predetermined distance, instead of close contact with the skin. When a moving body exists around the apparatus (for example, the hand is waved), there arises a problem in that the apparatus is affected by disturbance due to this, and it is difficult to perform a correct measurement.

### SUMMARY

The presently disclosed subject matter may provide an apparatus and a method in which the burden on the subject is small and which are hardly affected by disturbance.

The blood flow measuring apparatus may comprise: a module which is configured to emit radiation to an object to be measured and receive reflection of the radiation, to detect movement of the object to be measured based on a Doppler effect; a reflective holding member which internally hold the module with a gap from an abutment surface that is to be butted against a skin, and which includes a reflective member covering the module and configured to reflect the radiation; and a process circuit which is configured to receive an output from the module, and which is configured to perform at least a process related to a blood flow speed.

The process circuit may perform frequency analysis on the blood flow speed to determine a degree of excitation of a subject.

According to an aspect of the presently subject matter, there is also provided a probe for measuring a blood flow. The probe may comprise: a module which is configured to emit radiation to an object to be measured and receive reflection of the radiation, to detect movement of the object to be measured based on a Doppler effect; and a reflective holding member which internally hold the module with a gap from an abutment surface that is to be butted against a skin, and which includes a reflective member covering the module and configured to reflect the radiation.

The reflective holding member may have a shape corresponding to a part of an ellipsoidal body that has a first focal point and a second focal point. The first focal point may be located in the part of the ellipsoidal body, and the second focal point may be located in the other of the ellipsoidal body. The module may be held in a vicinity of the first focal point in the part of the ellipsoidal body. The object to be measured may be located in a vicinity of the second focal point.

A diameter or length of the reflective holding member may be changeable to enable a position of the second focal point to be changed.

The part of the ellipsoidal body may be formed by combining a plurality of parts of ellipsoidal bodies that have third focal points and fourth focal points. The third focal points may correspond to the first focal point and be located at the same position. The fourth focal points may correspond to the second focal point and be located at different positions.

The module may include a microwave Doppler module.

The blood flow measuring method may comprise: providing a module which is configured to emit radiation to an object to be measured and receive reflection of the radiation, to detect movement of the object to be measured based on a Doppler effect, the module covered with a reflective member configured to reflect the radiation; and measuring a blood flow by the module covered with the reflective member.

### BRIEF DESCRIPTION OF THE DRAWINGS

FIGS. 1A and 1B are views showing the configuration of a blood flow measurement probe in an embodiment.

FIG. 2 is a functional block diagram of a blood flow measuring apparatus.

FIG. 3 is a view showing a usage situation of the blood flow measurement probe shown in FIGS. 1A and 1B.

FIGS. 4A and 4B are views showing the configuration of a blood flow measurement probe in another embodiment.

FIG. 5 is a view showing an embodiment in which the focal point on the human side is changeable.

FIG. 6 is a view showing an embodiment which is configured so as to have a plurality of focal points on the side of the human body.

FIG. 7 is a view showing an embodiment which is configured so as to have a focus position at a depth in a direction inclined with respect to the body surface.

FIGS. 8A and 8B are views showing temporal changes of a measured blood flow speed.

FIGS. 9A to 9C are views illustrating calculations of HF and LF.

FIGS. 10A and 10B are views showing an experimental example using a rat.

FIGS. 11A to 11C are views showing experimental data for determining an influence of disturbance.

### DETAILED DESCRIPTION OF EXEMPLARY EMBODIMENTS

### 1. First Embodiment

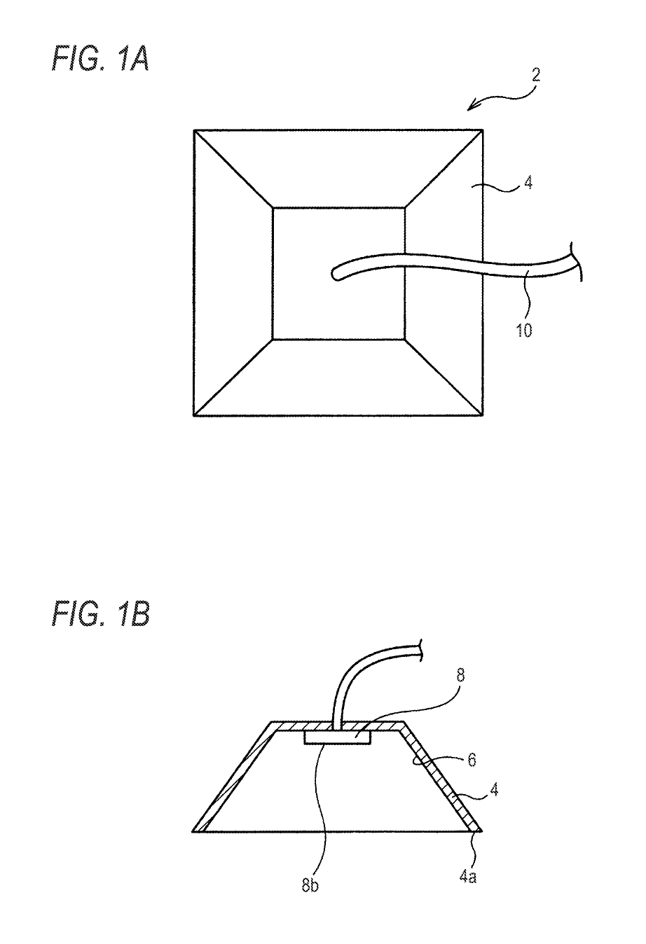

FIGS. 1A and 1B show the configuration of a blood flow measurement probe 2 in an embodiment of the presently disclosed subject matter. FIG. 1A is a plan view, and FIG. 1B is a side sectional view. A reflective holding member 4 which is made of a plastic material, and which has a truncated pyramidal shape has a hollowed interior. The lower surface of the reflective holding member 4 is opened. The lower portion of the reflective holding member 4 functions as a skin abutment surface 4a.

The inner surface of the reflective holding member 4 is plated with aluminum 6. A microwave Doppler sensor 8 is fixed to the inner side of the upper plate of the reflective holding member 4. Power input and signal output from the microwave Doppler sensor 8 are performed through a line 10.

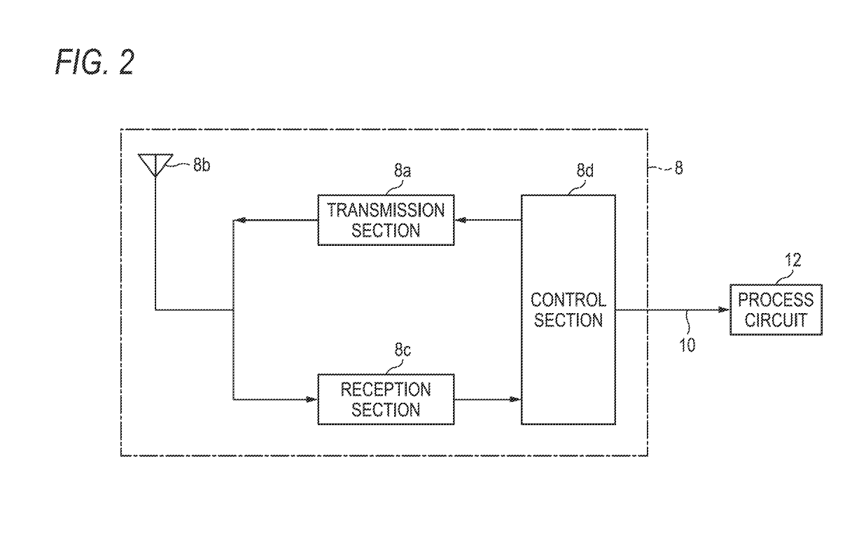

FIG. 2 is a block diagram of the whole blood flow measuring apparatus using the blood flow measurement probe 2 of FIGS. 1A and 1B. A transmission section 8a of the microwave Doppler sensor 8a causes a microwave (electromagnetic wave of, for example, 4.2 GHz) to be radiated from an antenna 8b. The microwave reaches the blood vessel to be measured, through the skin of the subject, and reflects from the object to be measured (blood flow). The reflection is received by a reception section 8c via the antenna 8b. A control section 8d controls the reception section 8c, and detects the phase difference between the transmitted and received waves, thereby calculating and outputting the speed of the object (blood flow).

The output speed signal is sent to a process circuit 12 via the line 10. The process circuit 12 performs a graph displaying process and pulsation detection based on the received speed signal.

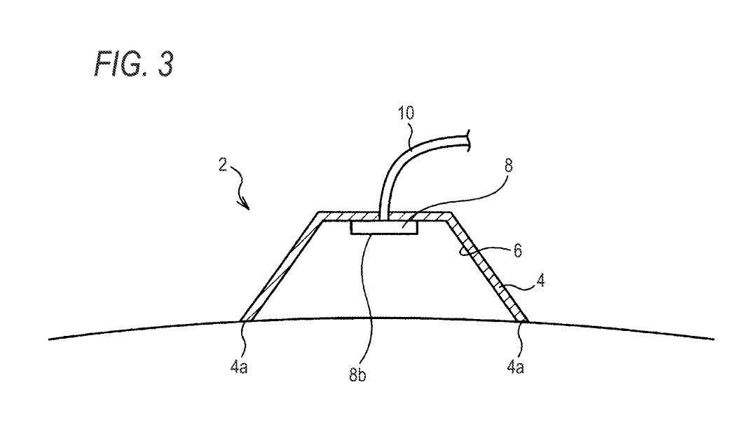

FIG. 3 shows a state in the case where the blood flow measurement probe 2 is butted against the human body and the blood flow is measured. As shown in the figure, the antenna 8b is disposed on the lower surface of the microwave Doppler sensor 8. The abutment surface 4a of the reflective holding member 4 is butted against the measurement portion of the human body 20. Preferably, the measurement portion is a portion (the abdominal aorta) of the descending aorta or the like which does not overlap with the heart, because a large amount of blood flows in the heart and the measurement is hardly performed, and further because a change in the blood flow speed does not remarkably appear in a blood vessel which is largely separated from the heart.

In the embodiment, since the inner surface of the reflective holding member 4 is plated with the aluminum 6, there is no possibility of receiving a reflected wave (noises) from a moving object other than the blood flow which is the object to be measured. This is because, since the microwave is reflected by the aluminum 6, the microwave is not radiated to a direction other than the desired direction (the direction toward the human body), and a microwave from a direction other than the desired direction is not received. Even when the palm is moved in the periphery of the probe during the measurement, for example, noises due to this are not received (even when such noises are received, the level is very low). Furthermore, the measurement is performed in a non-contact manner with respect to the blood flow, and therefore it is not affected by the contact impedance and polarization.

According to an aspect of the presently disclosed subject matter, an influence of disturbance is eliminated, and the blood flow can be measured more correctly.

### 2. Second Embodiment

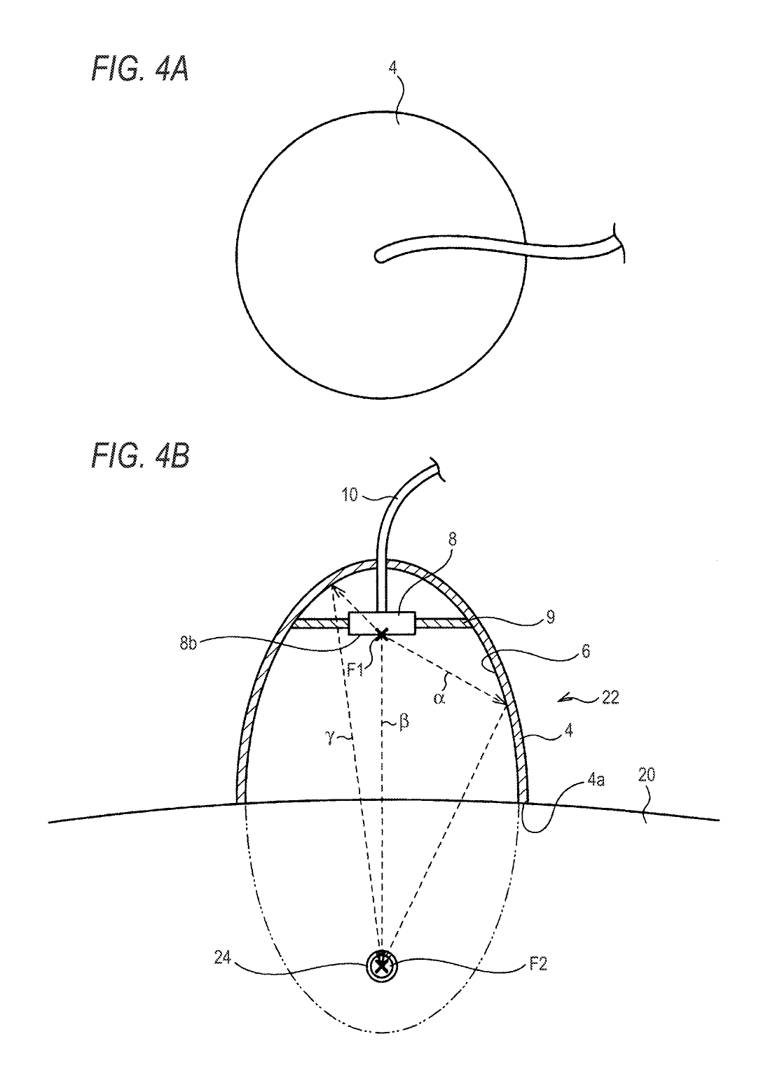

FIGS. 4A and 4B show the configuration of a blood flow measurement probe 22 in a second embodiment. FIG. 4A is a plan view, and FIG. 4B is a side sectional view. The portions corresponding to those of the blood flow measurement probe 2 of FIGS. 1A and 1B are denoted by the same reference numerals. The embodiment is configured in a similar manner as the first embodiment, but largely different in that the shape of the inner surface which is plated with the aluminum 6 constitutes a part of an ellipsoidal body.

The microwave Doppler sensor 8 is held to the reflective holding member 4 by a rod-like stay 9. The microwave Doppler sensor 8 is held so that the antenna 8b is located at one focal point F1 of the ellipsoidal body formed by the shape of the inner surface. On the other hand, the shape of the ellipsoidal body is designed so that the other focal point F2 of the ellipsoidal body is located at the position of the descending aorta 24 which is the object to be measured.

According to the configuration, external noises are prevented from entering, and moreover the microwave emitted in any direction reaches the descending aorta which is the object to be measured, as indicated by α, β, and γ in FIG. 4B. Similarly, all reflected waves which are reflected from the descending aorta in a predetermined angular range are received by the antenna 8b of the microwave Doppler sensor 8. Therefore, the measurement accuracy can be enhanced.

According to as aspect of the presently disclosed subject matter, the sensitivity can be further enhanced.

### 3. Other Embodiments

(1) In the above-described embodiments, the inner surface of the plastic member is plated with the aluminum 6. The inner surface may be plated with any material other than aluminum as far as the material reflects a microwave. Alternatively, vapor deposition or pasting may be performed in place of plating. These materials may be disposed on the outer surface or intermediate portion of the reflective holding member 4.

Alternatively, the reflective holding member 4 itself may be configured by a material which reflects a microwave, such as aluminum.

(2) in the embodiments, the measurement is performed while radiating a microwave. Alternatively, an electromagnetic wave of another frequency, an ultrasonic wave, or light may be radiated. In the alternative, a reflective material which is adapted to the kind of radiation is preferably used.

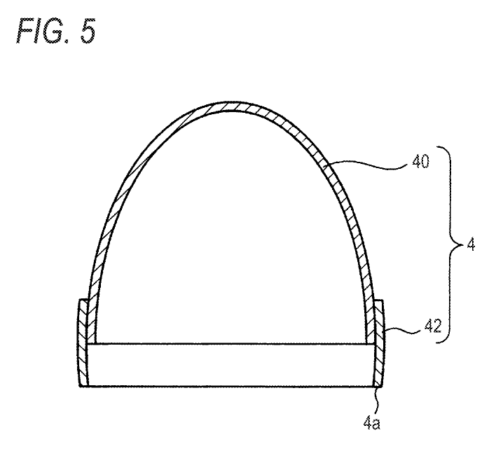

(3) In the embodiments, it is assumed that the distance from the human body to the microwave Doppler sensor 8 is predetermined. Alternatively, the position of the focal point may be changeable depending on the object to be measured. As shown in FIG. 5, for example, a second reflective holding member 42 which can be vertically slidably adjusted with respect to a first reflective holding member 40 may be disposed, and these members may configure the reflective holding member. When the second reflective holding member 42 is vertically adjusted, the focal point can be moved so as to be adapted to the object to be measured. Alternatively, the diameter may be changed in place of the length.

According to an aspect of the presently disclosed subject matter, the focal point can be changed in accordance with the object to be measured, and a more sensitive measurement can be performed.

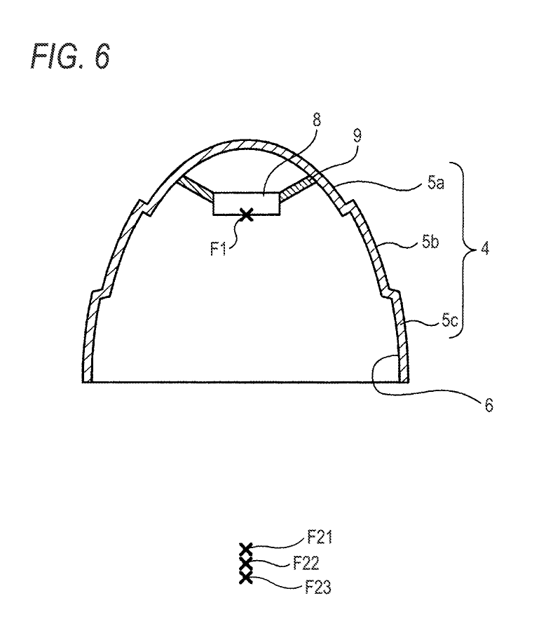

(4) As shown in FIG. 6, parts 5a, 5b, 5c of a plurality of ellipsoidal bodies which share the one focal point F1 may be combined with one another to configure the reflective holding member 4. According to the configuration, objects to be measured which are located respectively at three positions F21, F22, F23 can be measured.

According to an aspect of the presently disclosed subject matter, a measurement can be performed with a high sensitivity on all of objects to be measured which are at different positions.

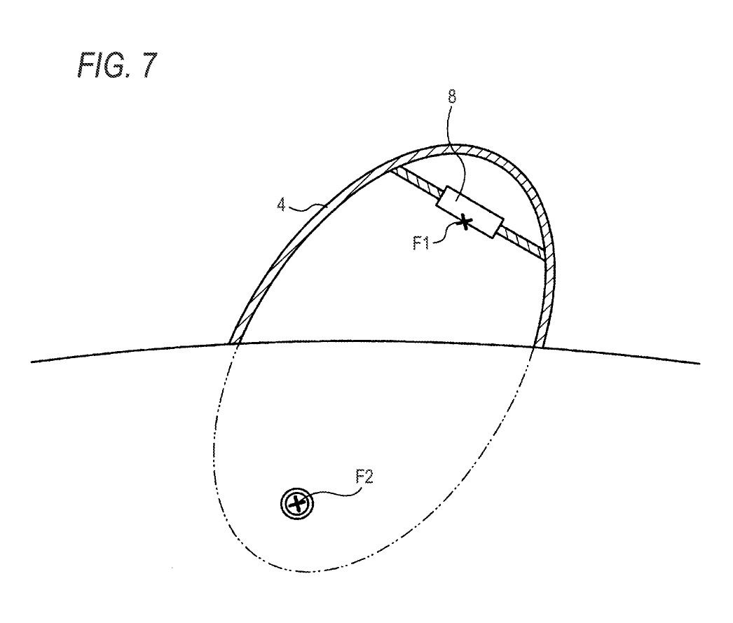

(5) in the embodiments, the reflective holding member 4 is configured by dividing an ellipsoidal body in a substantially middle thereof in parallel to the minor axis. As shown in FIG. 7, alternatively, the reflective holding member 4 may be configured by dividing an ellipsoidal body by a plane which forms a certain angle with respect to the minor axis. According to the configuration, the directionality can be inclinedly provided, so that the measurement is enabled even in the case where a portion which reflects a microwave exists immediately above the object to be measured.

### 4. Detail of Process Circuit 12

The process circuit 12 receives the speed signal from the microwave Doppler sensor 8, and can perform various processes. Hereinafter, some examples of the processes will be shown.

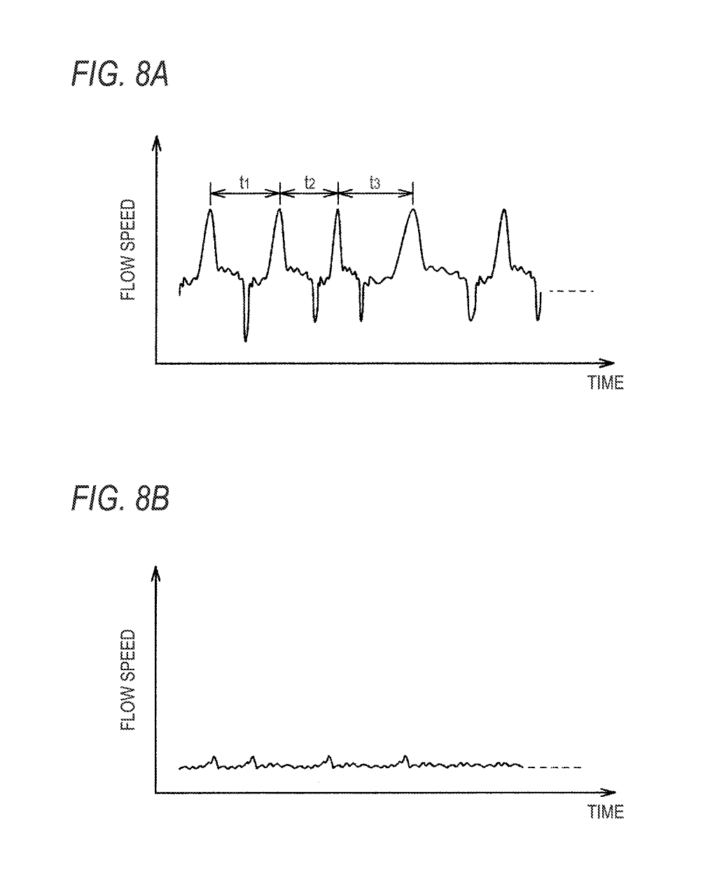

According to the blood flow measuring apparatus of the presently disclosed subject matter, it is possible to check the existence or non-existence of a blood flow, and to determine the necessity for cardiac massage, or the like. In this case, the process circuit 12 produces a graph showing the temporal transition of the blood flow speed, and displays it on a display device or the like. FIGS. 8A and 8B show display examples. As compared to the case where there, as shown in FIG. 8A, are large changes (pulsations) in the blood flow speed and the blood is properly ejected from the heart, in the case where the flow speed is low and constant as shown in FIG. 8B, it is possible to determine that the blood is not ejected. Therefore, the doctor can know that a treatment such as cardiac massage is necessary. This can be known also by monitoring an electrocardiogram. However, there is a case where, despite that the heart operates, the blood is not ejected (because the heart improperly operates). Therefore, it is preferable to directly monitor the blood flow.

Moreover, it is possible also to measure the degree of excitation. In this case, the process circuit 12 calculates the pulsation intervals based on the temporal change of the blood flow speed. In the case of FIG. 8A, for example, intervals between adjacent peaks t1, t2, t3, . . . are pulsation intervals. The degree of excitation can be obtained from the degree of fluctuation of the pulsation intervals. Specifically, when the following process is executed by a CPU of the process circuit 12 in accordance with a program, it is possible to obtain the degree of excitation.

According to an aspect of the presently disclosed subject matter, the degree of excitation of the subject can be easily acquired.

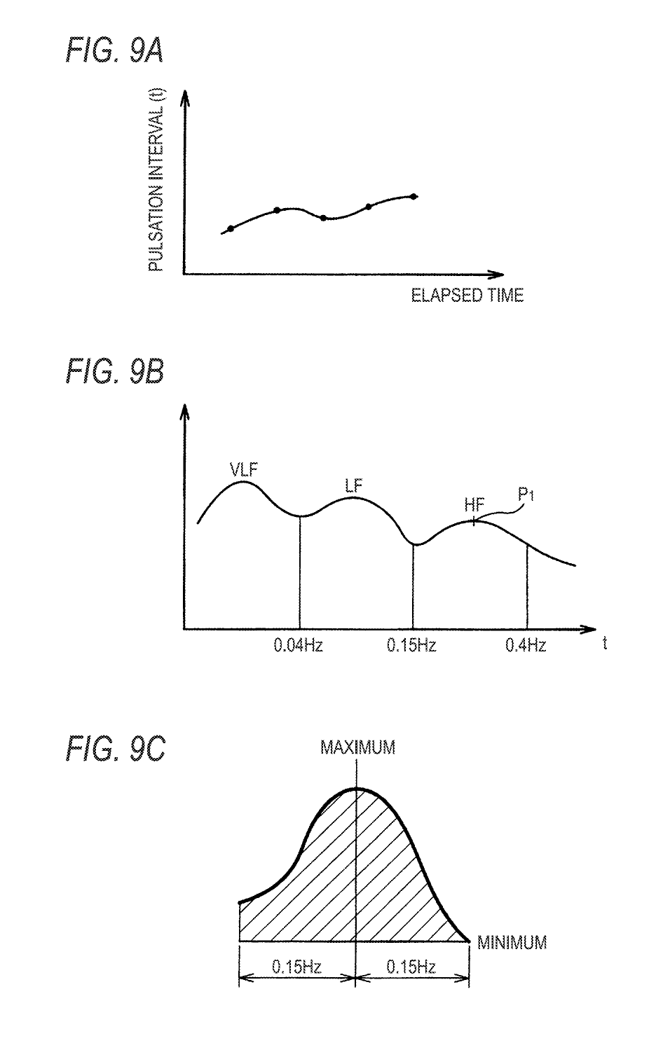

First, the CPU calculates the temporal change of the pulsation intervals and plots them (see FIG. 9A). The time intervals of the plot with respect to the abscissa is made corresponding to the actual one pulsation period. The temporal change of the pulsation intervals is a discrete value for each pulsation. As shown in FIG. 9A, therefore, they are connected to one another with a smooth curve by spline interpolation. As a result, the waveform of the pulsation interval variation can be obtained.

Next, the CPU performs resampling at time intervals (for example, several tens of ms) which is shorter than one pulsation, based on the produced waveform of the pulsation interval variation, thereby obtaining time-series data of the pulsation intervals. The time-series data are frequency analyzed, and values for respective frequency components are calculated. The value obtained by the frequency analysis is calculated for each unit time interval of the resampling.

FIG. 9B shows the waveform of the thus obtained frequency analysis. The ordinate indicates the power spectral density (the unit: msec²·Hz), and the abscissa indicates the frequency (the unit: Hz). The wave having a peak which appears in a low frequency is called VLF, that having the next peak is called LF, and that having the further next peak is called HF.

Then, the CPU calculates the HF value in the following manner. First, the maximum value in the range of 0.15 Hz to 0.4 Hz (alternatively, the range may be extended to 2 Hz) is found (see P₁ in FIG. 9B). As shown in FIG. 9C, then, the waveform in the 0.15 Hz zone around the maximum value is extracted, and its area is calculated while the minimum value is set as the baseline. The area is divided by the frequency width (0.3 Hz) to calculate the average value. The average value is the value of the pulsation interval HF.

The CPU calculates and records a 5-second average value of the values of the pulsation interval HF which are calculated for respective unit time intervals of the resampling.

The CPU calculates also the value of the pulsation interval LF in a similar manner as described above.

The CPU calculates pulsation interval LF/pulsation interval HF, whereby the degree of excitation can be obtained. When the thus calculated degree of excitation is given as information to a game machine or the like, for example, it is possible to realize a game machine or the like in which the story line is changed depending on the degree of excitation. According to the presently disclosed subject matter, an advantage is provided that, without requiring adhesion of electrodes or the like, the blood flow speed can be measured simply by butting the blood flow measurement probe against the human body.

Moreover, the presently disclosed subject matter can be applied to a sleep preventing system for a driver of a vehicle or the like by using a phenomenon that HF is lowered during sleep.

When the measurement is performed while the depth of the other focal point is gradually change (for example, by using the blood flow measurement probe 22 having a structure such as shown in FIG. 5), furthermore, a stereoscopic image of the blood flow can be reconstructed.

### EXAMPLES

### 1. Experiment 1

An experiment was conducted in order to show that the blood flow speed can be measured by using the microwave Doppler sensor 8.

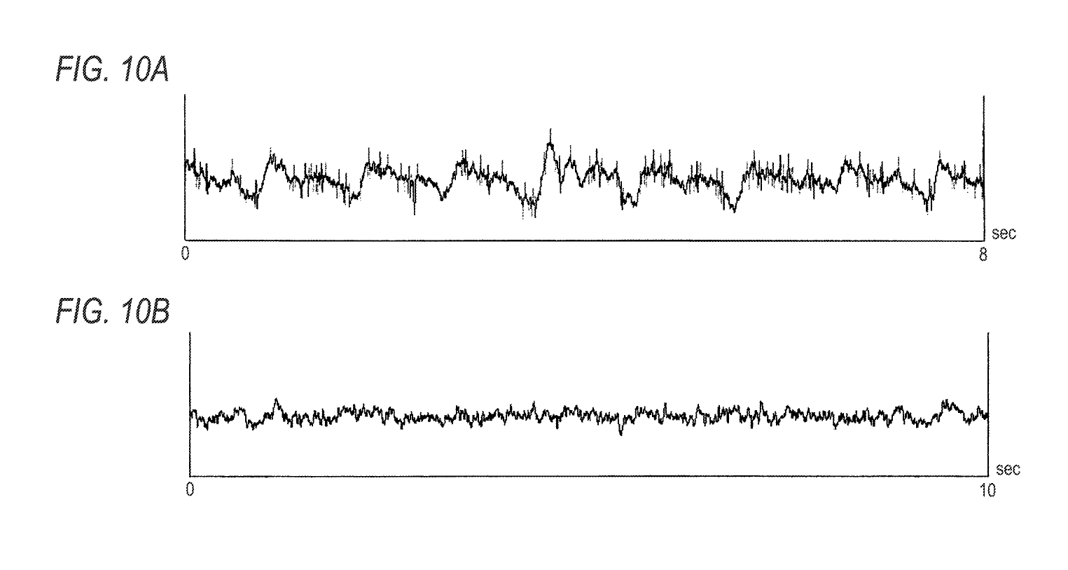

A cannula in which one end was inserted into the hip artery of an anesthetized rat was outward derived, and the other end was inserted into the cervical artery. Therefore, a blood flow is produced in the cannula. A polyethylene tube having a strength at which physical deformation is not caused by the blood pressure was used in the cannula in order to prevent a physical change of the cannula itself from being measured. FIG. 10A shows a temporal change of the output of the microwave Doppler sensor 8 in the case where the blood flow measurement probe 22 was approached toward the cannula. It is seen that the pulsation was able to be recognized and the blood flow speed was measured.

FIG. 10B shows a temporal change of the output of the microwave Doppler sensor 8 in the case where the cannula was removed away from the above-described configuration.

### 2. Experiment 2

An experiment on the influence of disturbance was conducted by using the blood flow measurement probe 22 shown in FIGS. 4A and 4B (a probe same as that of Experiment 1 was used as the microwave Doppler sensor 8). The reflective holding member 4 having a height of about 20 cm and a diameter of about 15 cm was used. The reflective holding member 4 which itself is formed by a metal was used. The object to be measured was the descending aorta, and a measurement was performed while the blood flow measurement probe 22 was butted against an abdominal portion.

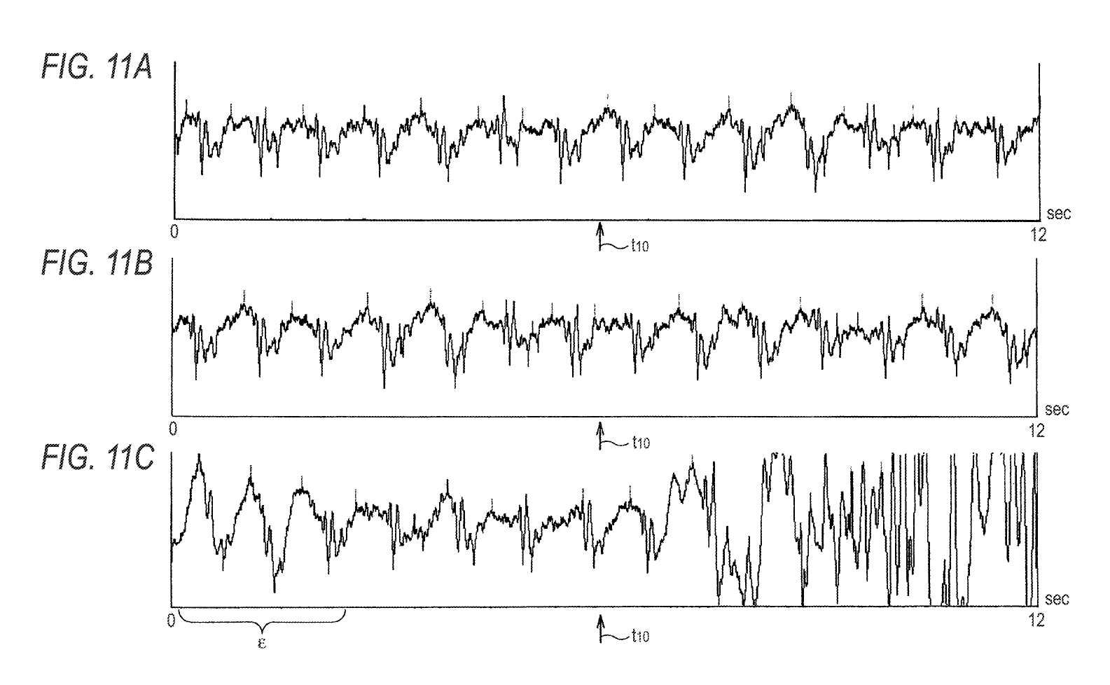

FIGS. 11A and 11B show measurement results, and FIG. 11C shows a measurement result in the case where the microwave Doppler sensor 8 was not covered by the reflective holding member 4 so as to be exposed to the exterior. In the measurements of FIGS. 11B and 11C, a person other than the subject moved the hand in front of the blood flow measurement probe 22 (i.e., in rear of the subject) during a period from timing t₁₀ to the end of the graph. As apparent from comparison of the graphs, it is clear that the case where the sensor is covered by the reflective holding member 4 is more insusceptible to large disturbance in which the hand is moved. Moreover, it is obvious that the measurement was not affected by disturbance also in the state where distinct disturbance in which the hand was moved was not produced (see the region ε).

In the measurement of FIG. 11A, a person other than the subject moved the hand in rear of the blood flow measurement probe 22 (in front of the subject) during the period from timing t₁₀ to the end of the graph. Also in this case, an influence of disturbance was not caused because the sensor was covered by the reflective holding member 4.

What is claimed is:

1. A blood flow measuring apparatus comprising:
a module which is configured to emit radiation to an object to be measured and receive reflection of the radiation, to detect movement of the object to be measured based on a Doppler effect;
a reflective holding member which internally hold the module with a gap from an abutment surface that is to be butted against a skin, and which includes a reflective member covering the module and configured to reflect the radiation; and
a process circuit which is configured to receive an output from the module, and which is configured to perform at least a process related to a blood flow speed.

2. The blood flow measuring apparatus according to claim 1, wherein
the process circuit performs frequency analysis on the blood flow speed to determine a degree of excitation of a subject.

3. The blood flow measuring apparatus according to claim 1, wherein
the reflective holding member has a shape corresponding to a part of an ellipsoidal body that has a first focal point and a second focal point,
the first focal point is located in the part of the ellipsoidal body, and the second focal point is located in the other of the ellipsoidal body,
the module is held in a vicinity of the first focal point in the part of the ellipsoidal body, and
the object to be measured is located in a vicinity of the second focal point.

4. The blood flow measuring apparatus according to claim 3, wherein
a diameter or length of the reflective holding member is changeable to enable a position of the second focal point to be changed.

5. The blood flow measuring apparatus according to claim 3, wherein
the part of the ellipsoidal body is formed by combining a plurality of parts of ellipsoidal bodies that have third focal points and fourth focal points,
the third focal points correspond to the first focal point and are located at the same position, and
the fourth focal points correspond to the second focal point and are located at different positions.

6. The blood flow measuring apparatus according to claim 1, wherein
the module includes a microwave Doppler module.

7. A probe for measuring a blood flow, the probe comprising:
a module which is configured to emit radiation to an object to be measured and receive reflection of the radiation, to detect movement of the object to be measured based on a Doppler effect; and
a reflective holding member which internally hold the module with a gap from an abutment surface that is to be butted against a skin, and which includes a reflective member covering the module and configured to reflect the radiation.

8. The blood flow measuring apparatus according to claim 7, wherein
the reflective holding member has a shape corresponding to a part of an ellipsoidal body that has a first focal point and a second focal point,
the first focal point is located in the part of the ellipsoidal body, and the second focal point is located in the other of the ellipsoidal body,
the module is held in a vicinity of the first focal point in the part of the ellipsoidal body, and
the object to be measured is located in a vicinity of the second focal point.

9. The blood flow measuring apparatus according to claim 8, wherein
a diameter or length of the reflective holding member is changeable to enable a position of the second focal point to be changed.

10. The blood flow measuring apparatus according to claim 8, wherein
the part of the ellipsoidal body is formed by combining a plurality of parts of ellipsoidal bodies that have third focal points and fourth focal points,
the third focal points correspond to the first focal point and are located at the same position, and
the fourth focal points correspond to the second focal point and are located at different positions.

11. The blood flow measuring apparatus according to claim 8, wherein
the module includes a microwave Doppler module.

12. A blood flow measuring method comprising:
providing a module which is configured to emit radiation to an object to be measured and receive reflection of the radiation, to detect movement of the object to be measured based on a Doppler effect, the module covered with a reflective member configured to reflect the radiation; and
measuring a blood flow by the module covered with the reflective member.
